(19) United States
(12) Patent Application Publication
(10) Pub. No.: US 2006/0127411 A1
(43) Pub. Date: Jun. 15, 2006

(54) Immune -modulating peptide made of s.aureus enterotoxin b

(75) Inventor: Karsten Neuber, Hamburg (DE)

Correspondence Address: MCDERMOTT, WILL & EMERY, 4370 LA JOLLA VILLAGE DRIVE, SUITE 700, SAN DIEGO, CA, 92122, US

(73) Assignee: BioAgency AG

(21) Appl. No.: 10/504,793

(22) PCT Filed: Feb. 14, 2003
(86) PCT No.: PCT/EP03/01511

§ 371 Date: May 6, 2005

(30) Foreign Application Priority Data
Feb. 15, 2002 (DE) 102077347
Sep. 4, 2002 (DE) 102408661

(51) Int. Cl. A61K 39/02; C07H 21/04; C12P 21/06; C07K 14/315; C12N 15/74

(52) U.S. Cl. 424/190.1; 530/350; 435/69.1; 435/252.3; 435/471; 536/23.7

(57) ABSTRACT

The invention relates particularly to peptides which are specifically capable of binding IgE antibodies and can be obtained from naturally occurring S. aureus enterotoxin B (SEB), for example. The immune-modulating properties thereof are substantially different from those of bacterial SEB. Surprisingly, the inventive peptides do not induce proliferation of T cells, as opposed to SEB. Due to their properties, said peptides are suitable for treating diseases that are characterized by an increased serum IgE level and/or an increased production of interferon gamma and for treating diseases that are characterized by an imbalance in the Th1 and Th2 response, e.g. atopic eczema, lupus erythematosus, Crohn's disease, multiple sclerosis, psoriasis, and rheumatoid arthritis.

The invention relates particularly to peptides which are specifically capable of binding IgE antibodies and are obtainable, for example, from naturally occurring S. aureus enterotoxin B (SEB). The immune-modulating properties thereof are substantially different from those of bacterial SEB. Surprisingly, the inventive peptides do not induce proliferation of T cells, as opposed to SEB. Due to their properties, the peptides are suitable for treating diseases that are characterised by an increased serum IgE level and/or an increased production of interferon gamma and for treating diseases that are characterised by an imbalance in the Th1 and Th2 immune response, e.g. atopic eczema, lupus erythematosus, Crohn's disease, multiple sclerosis, psoriasis and rheumatoid arthritis.

Staphylococcus aureus is a wide-spread pathogenic microorganism capable of triggering a number of serious infectious diseases. S. aureus generates enterotoxins (enterotoxin A, B, C, D, E and TSST-1) and endotoxins (coagulase, staphylocinase, lipase, hyaluronidase, DNAse). As a result of the development of multiple resistances to different antibiotics, S. aureus has acquired great importance in the last few years as a pathogen causing infections in hospitals.

The enterotoxins (SE) generated by S. aureus can cause food poisoning and septic shock, among others, in man (Marrack, P. & Kappler, J. (1990), Science 248, 705).

These are medium-sized proteins (20-30 kD) which resemble each other closely regarding their amino acid sequences. There is a more than 90% similarity between Staphylococcus enterotoxin A (SEA) and Staphylococcus enterotoxin E (SEE) (Marrack, P. & Kappler, J. (1990), Science 248, 705).

The interest in the immunological properties of these toxins has risen substantially since it has been discovered that, even in concentrations of 10⁻¹³ M-10⁻¹⁶ M, they are among the strongest inducers of lymphocyte proliferation and this proliferation is restricted to T cells (Fleischer, B. & Schrezenmeier, H. (1988), J. Exp. Med. 167, 1697).

The exact clarification of the stimulation mechanisms began with the observation that SEs do not stimulate highly purified T cells and that the toxin-induced proliferation depends on the presence of accessory cells, monocytes or B lymphocytes.

However, in contrast to normal antigens, the enterotoxins are not processed and presented in the antigen binding site of the MHC-II molecule but they crosslink the MHC-II complex on monocytes or B cells with the T cell receptor (Herrmann, T. et al. (1992), Eur. J. Immunol. 2, 1935).

The binding affinity of the enterotoxins to different MHC-II proteins of man varies. Most SEs preferably bind to HLA-DR, less strongly to DQ and almost not at all to DP (Fleischer, B. (1991), Immun. Infekt. 19, 8). The reason for the strong T cell stimulation which, however, is limited in contrast to lectins such as concanavalin A, lies in the specific binding of the SEs to the outside of the V_{β} chain of the T cell receptor (TCR). The other components of the T cell receptor, the so-called V_{β}, J or D segments do not form binding sites for SEs. Since only T lymphocytes with a certain V_{β} chain of the TCR are stimulated by certain SEs, the toxins are referred to as antigens rather than mitogens.

However, since the number of circulating T cells which can be stimulated by a certain toxin is much greater than the number of the T lymphocytes specific for a conventional antigen, the SEs are referred to as “superantigens” (Marrack, P. & Kappler, J. (1990), Science 248, 705).

Depending on the frequency of the V_{β} population, 5-30% of the circulating T cell repertoire can be stimulated in vivo by bacterial superantigens (Choi, Y. (1990), J. Exp. Med. 172, 981). This T cell stimulation induces a multitude of complex pathophysiological mechanisms which have not yet been explained completely so far. In the animal model, but also in the case of man, intoxication with superantigens leads to systemic reactions which can range from fever to cardiovascular shock (Fleischer, B. (1991), Immun. Infekt. 19, 8). Table 1 shows the immunological reactions induced by enterotoxins of S. aureus. These reactions are likely to be triggered by complex immunological processes. Binding of the toxins to the MHC-II complex and TCR leads to proliferation, liberation of cytokines and other mediators and to antibody generation (Table 1). In addition, the cutaneous lymphocyte-associated antigen (CLA), which controls the recirculation of T cells into the skin, is expressed more strongly (Zollner, T. M. (1996), Immunol. Letters 49, 111).

TABLE 1
Immunological reactions induced by S. aureus enterotoxins.

| Toxin | Immunological effect |
|---|---|
| SEB | Activation of suppressor cells |
| SEA-SEE | Antibody generation |
| SEA, SEB, TSST-1 | Cytotoxicity and antitumour effect |
| | Cytokine synthesis |
| SEB, TSST-1 | IL-1 |
| SEA, SEB, SEC, TSST-1 | IL-2 |
| SEA, SEB | IL-4 |
| SEA, SEB, TSST-1 | IFNγ |
| SEA, SEB, SEC1, TSST-1 | TNF |
| SEA, SEB | Stimulation of NK cells |
| SEA, SEB, SED | T cell anergy |
| SEB | Apoptosis |

After this hyper-reactive phase, the superantigen-stimulated T cells change into an anergic state such that a strong immunosuppression can occur. On the other hand, autoreactive but anergic T cells can be activated which then trigger autoimmune diseases. In the case of multiple sclerosis (Hafler, D. A. (1988), J. Exp. Med. 167, 1313) and rheumatoid arthritis (Palliard, X. (1991), Science 253, 325) an oligoclonal selection of certain V_{β} chains has already been detected which points towards a superantigen-mediated pathomechanism of these diseases.

In addition, it has been possible to show that SEB increases the IgE synthesis and the production of interleukin-4 in patients with atopic eczema (Neuber, K, (1993), Hautarzt 44, 135; Ring, J. et al. (1992), Allergy 47, 265; Neuber, K. et al. (1995), Int. Arch. Allergy Immunol. 107, 179). Moreover, an eczema-triggering effect of SEB has been detected in the epicutaneous test (Hofer, M. F. (1999), J. Invest. Dermatol. 112, 171).

Like allergic rhinoconjunctivitis and allergic bronchial asthma, atopic eczema belongs to the diseases in the case of which a pathologically increased IgE synthesis occurs. So far, it has been possible to treat these diseases only symptomatically, i.e. the suppression of the IgE synthesis takes place non-specifically by suppressing the T lymphocytes participating in the synthesis. The drugs most frequently used systemically in this case are cortisone or cyclosporin A. Frequently, these substances have serious systemic side effects (skin atrophy, diabetes, hypertension, kidney toxicity and hepatotoxicity, among others). By a specific modulation of the excessively increased IgE synthesis, such serious side effects would be avoidable. The same applies also to diseases which are accompanied by an increased interferon-gamma synthesis of the lymphocytes. A good example of such a state is psoriasis vulgaris. In the case of this disease, the T cells produce increased amounts of interferon-gamma and in this way induce the hyperproliferation of the epidermis. Immunosuppressives such as cortisone or cyclosporin A are also used for the therapy of psoriasis. The selective inhibition of cytokine production would substantially reduce the rate of side effects also in this case.

In a number of diseases, a pathological Th1 or a reduced Th2 reaction has been shown or is being considered as the causes (compare also Table 2). The results of animal experiments suggest that, in the case of these diseases, a change in the immune response in the direction of Th2 has a protective effect. Organ-specific autoimmune diseases deserve to be mentioned here such as multiple sclerosis (Shevach, E. M. et al. (1999), Springer Semin. Immunopathol. 21,249-262; Leonard, J. P. et al. (1997), Crit. Rev. Immunol. 17, 545-553), autoimmune uveitis (Singh, V. K. et al. (1999), Immunol. Res. 20, 147-161; Sun, B. et al. (1999), Int. Immunol. 11, 1307-1312; Egwuagu, C. E. et al. (1999), J. Immunol. 162, 510-517), diabetes mellitus requiring insulin (Rabinovitch, A. et al. (1998), Biochem. Pharmacol. 55, 1139-1149), rheumatoid arthritis (Muller, B. et at. (1998), Springer Semin. Immunopathol. 20, 181-196), Behcet's syndrome (Frassanito, M. A. et al. (1999), Arthritis Rheum. 42, 1967-1974) and also the helicobacter pylori infection (the cause of stomach ulcers and atrophic gastritis, among others) (Smythies, L. E. et al. (2000), J. Immunol. 165, 1022-1029; Fox, J. G. et al. (2000), Nat. Med. 6, 536-542; Mattapallil, J. J. et al. (2000), Gastroenterology 118, 307-315), inflammatory intestinal diseases such as Crohn's and other diseases (Romagnani, P. et al. (1997), Curr. Opin. Immunol. 9, 793-799; MacDonald, T. T. (1999), Curr. Top. Microbiool. Immunol. 236, 113-135), the acute organ transplant rejection reaction (Morelli, A. E. et al. (2000), Transplanation 69, 2647-2657) and spontaneous recurrent miscarriages (Jenkins, C. et al. (2000), Fertil. Steril. 73, 1206-1208).

Consequently, the task of the present invention consists of providing a new active principle which is suitable for treating diseases which are characterised by a raised IgE level and/or increased interferon-gamma production and which active principle does not exhibit the disadvantages known in the art. In particular, it is a task of the invention to provide active principles which are suitable for the therapy of diseases which are characterised by an imbalance in the ratio of the Th1 and the Th2 immune response.

Within the framework of the present invention, it has now been found surprisingly enough that peptides capable of achieving the above-mentioned tasks can be isolated from naturally occurring S. aureus enterotoxin B (SEB).

In particular, it is possible to isolate a peptide which is capable of binding specific IgE antibodies. In this respect, its immune-modulatory properties differ surprisingly enough considerably from those of bacterial SEB. The peptide according to the invention whose amino acid sequence is represented in SEQ ID NO: 10 surprisingly enough does not induce any proliferation of T cells, in contrast to SEB, but on the other hand inhibits the interferon-gamma synthesis of stimulated T lymphocytes. Due to its properties, the peptide is suitable for the therapy of diseases which are characterised by an increased serum IgE level and/or increased production of interferon-gamma. Preferably, these diseases consist of allergic rhinoconjunctivitis, allergic bronchial asthma and psoriasis vulgaris.

Apart from the peptide according to the invention, further peptides according to the invention have been produced which exhibit the same or essentially the same biological and/or immunological properties. Due to their particular properties, i.e. binding of IgE antibodies and inhibition of interferon-gamma synthesis without induction of T cell proliferation, the peptides according to the invention are particularly suitable for treating diseases which are associated with increased IgE synthesis and increased interferon-gamma synthesis such as for example in the case of atopic eczema (neurodermatitis). This state has been described in the case of chronic forms of atopic eczema. In this case, interferon-gamma producing T lymphocytes are present on an increased scale in the skin as well as increased serum IgE values. The therapeutic effect of the peptides may take place either directly by inactivating binding to the IgE or indirectly by modulation of the cytokine production of the T lymphocytes. In this connection, the inhibition of further cytokines such as IL-4 and IL-13 needs to be considered as well which are known to stimulate IgE synthesis.

The subject matter of the present invention is therefore a polypeptide which exhibits the N-terminal amino acid sequence according to SEQ ID NO:15 and is obtainable as 12 kD cleavage fragment by trypsin cleavage of S. aureus enterotoxin B.

The invention relates in particular to a polypeptide (in the following referred to as peptide P1) which exhibits the amino acid sequence indicated in SEQ ID NO: 10 and which inhibits the synthesis of interferon-gamma by stimulated T lymphocytes in vitro.

Moreover, the present invention comprises homologues and derivatives of the above-mentioned polypeptides which bind to IgE. The homologous polypeptides are those substances which, however, differ from the above-mentioned amino acid sequences, in particular from the sequence according to SEQ ID NO: 10, as a result of the exchange of one or several amino acids or by insertion or deletion of one or several amino acids, these having a sequence homology of approximately 50 to 99%, preferably of at least approximately 75%. A sequence homology of at least 85% is particularly preferred. According to the invention, a derivative should be understood to mean those polypeptides in the case of which one or several amino acids of the above-mentioned sequences, in particular of the sequence according to SEQ ID NO: 10, are replaced by a stereoisomer (i.e. replacement of one or several L-amino acids by D-amino acids) or a corresponding modified amino acid. Modified amino acids should be understood to mean those amino acids whose side chains have been chemically modified in comparison with naturally occurring amino acids, e.g. by glycosylation, phosphorylation, methylation etc. Such modifications are well known to a person skilled in art. According to a preferred embodiment of the invention, the homologues and derivatives binding IgE have the same or essentially the same properties as the above-mentioned peptide according to SEQ ID NO: 10, i.e. they inhibit the synthesis of interferon-gamma stimulated T lymphocytes and/or they do not induce the proliferation of T lymphocytes, in vitro.

According to a preferred embodiment of the invention, the homologue (in the following referred to as peptide P2) exhibits the amino acid sequence indicated in SEQ ID NO: 11.

The invention also includes polypeptides with a length of more than 9 amino acids which contain the amino acid sequence indicated in SEQ ID NO: 10 or 11 and inhibit the synthesis of interferon-gamma by stimulated T lymphocytes in vitro.

Moreover, the invention relates to a nucleic acid molecule which contains a nucleic acid sequence that codes for an above-mentioned polypeptide. Preferably, it has the nucleic acid sequence indicated in SEQ ID NO: 9. The nucleic acid molecule can be used in order to clone the sequence into an expression vector and to recombinantly produce the peptide according to the invention. These methods are well known to the person skilled in the art.

Within the framework of the present invention it has, surprisingly enough, been found that the peptides according to the invention, in particular P1 and P2, are capable of influencing the ratio between Th1 and Th2 cells and consequently also the ratio of the corresponding cytokines.

TABLE 2
Diseases with an imbalance in the Th1 / Th2 ratio which are suitable
for therapy with the peptides.

| Th1 preponderance | Th2 preponderance |
|---|---|
| Psoriasis vulgaris | Atopic eczema |
| Autoimmune uveitis | Allergic asthma |
| Allergic contact eczema | Allergic rhinoconjunctivitis |
| Behcet's syndrome | Ulcerative colitis |
| Diabetes mellitus | Basedow's disease |
| Hashimoto's disease | Multiple sclerosis (myelin |
| | reactive autoantibody) |
| Heliobacter pylori infection | Alibert's disease |
| Lupus erythematosus | |
| Crohn's disease | |
| Multiple sclerosis (activation of astrocytes) | |
| Organ transplant rejection reaction | |
| Psoriasis anthropathica | |
| Rheumatoid arthritis | |
| Spontaneous recurrent miscarriages | |

As a result of the immunological properties of the proteins and polypeptides according to the invention, these are particularly suitable for the production of a pharmaceutical composition for inducing or increasing a Th1 or Th2 immune response by increasing the Th2 response and reducing the Th1 response and vice versa, in particular for the production of a pharmaceutical composition for treating diseases associated with a (predominantly) Th2 or Th1 immune response. Preferably, these diseases are diseases from the group consisting of multiple sclerosis, autoimmune uveitis, diabetes mellitus, rheumatoid arthritis, Behcet's syndrome, heliobacter pylori infection, inflammatory bowel diseases (in particular Crohn's disease), acute organ transplant rejection reaction and spontaneous recurring miscarriages.

The subject matter of the invention also consists of the use of a polypeptide which exhibits or contains the amino acid sequence according to SEQ ID NO: 10, for the production of a pharmaceutical composition for the immune modulation and inhibition of the cytokine production of lymphocytes, in particular T lymphocytes. Included is also the use of such a polypeptide for the production of a pharmaceutical composition for treating diseases which are associated with an increased production of cytokines (e.g. interferon-gamma) and/or an increased level of IgE antibodies. These diseases are in particular atopic eczema (in particular the chronic form), bronchial asthma, allergic rhinoconjunctivitis, psoriasis, rheumatoid arthritis or multiple sclerosis. Further diseases are detailed in Table 2. According to one particular embodiment, the cytokine is interferon-gamma, interleukin 4 (IL 4), interleukin 5 (IL 5), interleukin 10 (IL 10), interleukin 12 (IL 12) and/or interleukin 13 (IL 13).

The present invention includes the use of one of the polypeptides described above which exhibits an amino acid sequence homologous to SEQ ID NO: 10 or derived therefrom. As regards the homologous sequences or those derived therefrom, reference is made to the above definition of “homologues” and “derivatives”, those IgE-binding polypeptides being included in particular which do not induce the proliferation of T lymphocytes and/or inhibit the synthesis of interferon-gamma by stimulating T lymphocytes in vitro. According to a particular embodiment of the invention, the polypeptide used has the sequence according to SEQ ID NO: 11. Insofar as the invention relates to the use of polypeptides which contain the sequence according to SEQ ID NO: 10 or SEQ ID NO: 11 or sequences homologous thereto or derived therefrom with the same or essentially the same biological or immunological properties as part of a longer amino acid sequence, polypeptides with a length of up to 30 amino acids, in particular with a length of up to 15 amino acids, are preferred.

As a result of their particular biological properties, the polypeptides according to the invention are suitable for the in vitro inhibition of the interferon-gamma production in lymphocytes, in particular in human, peripheral T lymphocytes of the blood.

The peptides according to the invention modulate both the stimulated and the spontaneous cytokine production of Th1 and Th2 lymphocyte subpopulations. Regarding the direction of the peptide-induced effects, the endogenous activation status of the lymphocytes seems to play an important part. In patients with atopic eczema, an inhibition of the endogenous increased synthesis of all cytokines by the peptide P1 according to the invention is observed. In the meantime, it has become known that, in these patients, it is not only the Th2 cytokines which are elevated but that an increased IFNγ production can be observed particularly in the case of the chronic stationery form. The peptide P1 according to the invention, in particular, is capable not only of inhibiting the Th2 cytokine synthesis of the lymphocytes of these patients but also the IFNγ production, which is of considerable therapeutic relevance, e.g. by reducing the symptoms as far as healing.

In the case of psoriatics, the induction of a greatly increased release of IL-10 by the peptides according to the invention is of particular interest. There are different indications that IL-10 is capable of favourably influencing the clinical picture of psoriasis. The targeted effect of the peptides according to the invention on the IL-10 production of the lymphocytes of psoriatics is here of particular therapeutic significance in the interaction with the effects on the other cytokines, e.g. by reducing the symptoms as far as healing.

The differentiated effects of the peptides according to the invention on the spontaneous and/or endogenous cytokine synthesis in the case of different pathological activation states of the immune system show that the peptides exhibit an immune modulatory effect also vis-à-vis other diseases caused by changes in the Th1/Th2 balance (Table 2).

The invention also relates to a pharmaceutical composition which contains, as active principle, one or several polypeptides according to the invention and, if necessary, also one or several pharmaceutically compatible auxiliary agents and/or carrier substances.

The peptide isolated from SEB is obtainable by digesting enterotoxin B from S. aureus with trypsin and subsequently isolating the 12 kD fragment from the reaction batch. As an alternative, gene technology methods of production are obviously also possible by introducing the nucleic acid sequence shown in SEQ ID NO: 9 into an expression vector, introducing the latter into suitable host cells, cultivating the host cells under conditions suitable for protein expression and isolating the polypeptide expressed which is encoded by the nucleic acid sequence.

The invention will now be illustrated by way of examples, a sequence protocol and figures.

### EXAMPLES

### Example 1

### Immunoblot for the Detection of Specific IgE Antibodies Against SEB

The gel electrophoretic separation of proteins (SDS-PAGE) was carried out according to the Lughtenberg et al. method (Lughtenberg, B. (1975), FEBS Lett. 58, 254). 12.5% polyacrylamide separation gels were used. Standard protein mixtures (Biorad, MW standard, 203 kD-7.5 kD) were separated in parallel for the determination of the molecular weight. Staining of the proteins was done with Coomassie Blue G-250. In the collecting gel, 10 pockets were ready to accept the samples, the quantities of protein used being between 10 and 100 μg. The protein solution to be separated (20 μl) was mixed with 20 μl of sample buffer, introduced into the collecting gel pockets, and subsequently electrode buffer was layered on top. The electrophoresis was carried out in electrode buffer and at a constant current flow of 20 mA. Once the bromophenol blue band had reached the end of the separation gel (development time up to 2 h), the electrophoresis was terminated. The protein mixtures to be examined were separated by gel electrophoresis (SDS-PAGE) and transferred to nitrocellulose (pore size 0.45 μm) by the Western blot method. The transfer took place with cooling in an: electroblot chamber (BioRad, Munich) directly after gel electrophoresis for 18-20 h at a constant current intensity of 0.2-0.3 A.

The nitrocellulose blots were incubated with human serum for 4 h on a revolving table at RT (Dianova, Hamburg). Subsequently, the blots were washed five times and then incubated again for 4 h at RT with a secondary antibody against human IgE (anti-human IgE antibody, Serva, Heidelberg) and the bonding was made visible by enzymatic dye reaction.

The result of the immunoblot is illustrated in FIG. 1. By means of the immunoblot it was possible to detect specific IgE antibodies against SEB in the sera of patients with atopic eczema.

In the immunoblots, peroxidase-labelled antibodies were used. It should be pointed out that the result of the blot illustrated in FIG. 1 (scanned in) does not correspond to the actual colour variations recognisable with the naked eye. The bands recognisable as shadows are so weak on the original blot compared with the bands incubated with the sera of atopics that they have to be evaluated as non-specific reactions.

### Example 2

### Cleavage of SEB with Trypsin

SEB (100 μg/10 μl ) were digested enzymatically with 10 μl trypsin (4 mg/ml distilled water) for 18 h at 37° C. Subsequently, the solution was applied onto SDS gels and the corresponding cleavage products were separated off. The conditions for the gel electrophoresis corresponded to the conditions indicated in Example 1. The cleavage products were incubated in the immunoblot with sera from the patients with atopic eczema detailed in Example 1. Serum from healthy control persons was used as control. The result of the trypsin digestion is illustrated in FIG. 2.

Following trypsin cleavage, a cleavage product approximately 12 kD in size was obtained with which the IgE from the serum of the patients with atopic eczema formed a bond detectable in the immunoblot (FIG. 2). The detection took place with the antihuman IgE antibody (Serva, Heidelberg) as described in Example 1.

### Example 3

### Sequencing of the IgE Binding Cleave Product

The cleavage product with a molecular weight of approximately 12 kD identified as IgE-binding was eluted from the gel and subsequently sequenced mechanically according to the method of P Edman (Edman, P. (1950), Acta Chem. Scand. 4, 283), the N-terminal sequence KVTAQELDYL being determined. The sequence analysis thus gave a sequence which begins in position 181 (from the N-terminal end) of the SEB molecule. FIG. 3 shows the result of the sequence analysis.

### Example 4

### Synthesis of Short Peptides

Starting out from the partially sequenced sequence of the 12 kD cleavage product, several peptides with overlapping sequences were produced in front of and behind the partially sequenced region:

| | |
|---|---|
| (a) H N G N Q L D K Y R S I T V R V F E | (SEQ ID NO: 1) |
| (b) V R V F E D G K N L L S F D V Q T N K | (SEQ ID NO: 2) |
| (c) V Q T N K K K V T A Q E L D Y L T R H | (SEQ ID NO: 3) |
| (d) Y L T R H Y L V K N K K L Y E F N N S | (SEQ ID NO: 4) |
| (e) E F N N S P Y E T G Y I K F I E N E N | (SEQ ID NO: 5) |
| (f) I E N E N S F W Y D M M P A P G D K F D | (SEQ ID NO: 6) |
| (g) G D K F D Q S K Y L M M Y N D N K M V D | (SEQ ID NO: 7) |
| (h) N K M V D S K D V K I E V Y L T T K K K | (SEQ ID NO: 8) |

(Note: the ‘SEQ ID NO:’ used corresponds to the sequence number <400> according to WIPO Standard ST. 25)

The peptides with the sequences (a) to (h) were then tested as described in Example 2. Peptide (c), apart from the 12 kD cleavage product, was the only one to exhibit an IgE binding.

The sequenced amino acid sequence of the peptide (c) was compared by means of a databank (SwissProt) with other known molecules for possible homologies. In this connection, it was found that a homology of 89% exists between the IgE-binding sequence of this peptide and an amino acid sequence of the extracellular domain of the human low affine IgE receptor (CD23) (compare FIG. 4). By means of the Smith-Waterman algorithm, it was possible to calculate the homology between SEB and CD23 as being significant (p=0.03).

Subsequently, two further peptides were synthesised which exhibited the sequence QELDYLTRH (P1; SEQ ID NO: 10) and QEEDFLTLH (P2; SEQ ID NO: 11 corresponding to the homologous sequence section from the CD23 molecule).

These two peptides were then used in further experiments to stimulate the lymphocytes (compare Examples 6 and 7).

### Example 5

### Dot Blot for the Detection of the Binding of Serum IgE to the Peptides

By means of a dot blot it was possible to show that the two peptides according to the invention are capable of binding IgE antibodies from the serum of patients with atopic eczema. For this purpose, the sera of 5 different patients with atopic eczema were first pre-purified via protein G columns (1 ml, Pharmacia). The sera were pre-filtered on 0.8 μm syringe adapter. Subsequently, the column was coupled “drop to drop” with 10 ml of binding buffer (20 mM PBS, pH 7.0) and washed at a flow rate of 1 ml/min. Subsequently, 1 ml of serum was filtered in each case and the filtrate collected as soon as it had assumed a light yellow colour. It was washed with 5 ml of PBS and the residual filtrate was collected until it became clear. The filtered serum was diluted in 20 mM PBS in a ratio of 1:2.

To carry out the dotblot, 20 μg of the peptides P1 and P2 as well as 1 μg of SEB were placed onto a membrane and dried overnight. The membrane was blocked with 5% of skimmed milk/PBS Tween. Subsequently, the sera and a positive control (sheep anti-SEB IgG, 10 μl/ml, SERVA) were used and incubated at RT for 1 h. Subsequently, the membranes were washed three times. For the detection of the serum IgE antibodies, biotin-labelled anti-human IgE (Pharmingen) was incubated in a concentration of 4 μg/5 ml for 1 h at RT and the membrane was subsequently washed three times. Following further incubation for 1 h at RT with a second streptavidin-labelled antibody and three wash operations, the dye reaction was effected with BCIP and NBT as substrate. The results of the dot blot are illustrated in FIG. 5. It can be seen that both peptides according to the invention exhibit a weak but clearly detectable IgE binding with the sera 4, 5 and 6. All sera exhibited an IgE binding to SEB.

### Example 6

### Immune-Modulatory Properties of the Peptides

Peripheral blood lymphocytes (PBMC) from healthy voluntary donors were isolated via Ficoll gradients and cultivated in a concentration of 10⁶ cells/ml in RPMI 1640 medium+10% FCS (fetal calf serum). SEB (1 μg/10⁶ cells) and the peptide (5 μg/10⁶ cells) were used as stimuli.

The proliferation of the cells and the intracellular production of interferon-gamma and interleukin-4 were measured by flow cytometry after 2 days' incubation at 37° C. and 5% CO₂. After isolating the peripheral blood lymphocytes by Ficoll-Paque gradient centrifuging, the cell count was set at 2×10⁶ cells/ml. The cells were introduced into conical 15 ml polypropylene vessels and subsequently incubated for 2 days in the incubator. For the proliferation, the PBMC was incubated with 60 μM bromodeoxyuidine (BrdU) during the last 5 h of the incubation period. To measure the intracellular cytokines, Brefeldin A (BFA, 20 μl/1 ml PBMC) were added for the last 5 h. Subsequently, 20 mM EDTA (100 μl/1 ml PBMC) were added and subsequently 10 ml of cold PBS. Following centrifuging, 3 ml of “FACS lysing solution” (BD Bioscience, Heidelberg) were added to the cells and the “FACS permeabilising solution” (BD Bioscience, Heidelberg) after a wash step. Subsequently, anti-BrdU-FITC antibody with DNase (proliferation) or anti-cytokine antibody (intracellular cytokine determination) were added to the cell granules and incubated for 30 min. Subsequently, the cells were washed and fixed with 200 μl of 1% paraformaldehyde. The fluorescence was measured by means of the flow cytometer (FACScan, BD Bioscience) and evaluated.

The proliferation was determined by incorporating BrdU into lymphocytes in the flow cytometer. The incubation of the lymphocytes with different concentrations of the peptides P1 and P2 according to the invention did not bring to light any significant effects of the peptides on proliferation (FIG. 6A). The SEB-induced proliferation was modified substantially neither by the peptides alone (FIG. 6A) nor the co-stimulation with SEB+peptide P1 (FIG. 6B). An inhibition of the SEB-induced lymphocyte proliferation occurred only in high concentrations (FIG. 6B).

In the next step, the intracellular IFNγ (FIG. 7) and IL 4 production was investigated following stimulation with SEB and the peptides P1 and P2 according to the invention (FIG. 7). In this connection, it was found that P1 suppresses the production of IFNγ, a cytokine which mediates a number of immunological functions, more strongly than P2 as a function of the dosage (FIG. 7A), and that IL-4 (FIG. 7B) is stimulated as a function of the dosage. The lymphocytes examined in these experiments originated from healthy candidates. Correspondingly, the ratio between IFNγ and IL-4 was modified by the peptides as a function of the dosage in favour of the IL-4 producing Th2 lymphocytes (FIG. 7C).

### Example 7

### Effect of the Peptides on the Secretory Cytokine Production

Peripheral blood lymphocytes from healthy voluntary donors were isolated via a Ficoll gradient and absorbed, in a concentration of 10⁶ cells/ml, in RPMI 1640-medium+10% FCS (fetal calf serum). The cells were introduced into plates of 24 wells and incubated with the two peptides (200 μg/10⁶ cells) for 2 days in the incubator (37° C., 5% CO₂). Subsequently, the supernatant liquors were collected and initially frozen at −20° C. The human Th1 cytokines IL-2, IFNγ and TNFα as well as the Th2 cytokines IL-4, IL-5 and IL-10 were measured in the cell culture supernatant liquor using the cytometric bead array (CBA) from B&D (Heidelberg). The test was carried out exactly in line with the manufacturer's instructions.

In a first series of experiments, the effects of the peptides P1 and P2 according to the invention on the spontaneous cytokine secretion of peripheral blood lymphocytes of health candidates (n=3) were examined. FIG. 8 shows the effects on the Th1 cytokines (IL-2, IFNγ, TNFα) and on the Th2 cytokines (IL-4, IL-5, IL-10).

It was found that P1 inhibits above all the spontaneous production of the Th1 cytokines IL-2 and IFNγ, where as P2 inhibits TNFα more strongly. P2 induces the synthesis of Th2 cytokines (IL-4, IL-5, IL-10) in healthy candidates whereas P1 completely inhibits also the synthesis of IL-4 and IL-5.

The results of these investigations show that P1 and P2 influence the ratio of Th1 and Th2 cytokines.

Consequently, the same experiment was carried out in the next step using peripheral blood lymphocytes from patients with severe atopic eczema (Th2>Th1) or severe psoriasis vulgaris (Th1>Th2). The results are shown in FIGS. 9 and 10.

It came to light that in patients with atopic eczema or with psoriasis opposing effects occur in some cases on spontaneous cytokine secretion which has already been modified endogenously. In patients with atopic eczema (FIG. 9), the spontaneous production of all cytokines, i.e. both Th1 and Th2 cytokines, had substantially increased. P1 inhibits the increased cytokine production of all Th populations in the case of these patients, but in particular also the IFNγ synthesis case which is elevated in the case of chronic atopic eczema.

In patients with psoriasis vulgaris, a substantial stimulation of the IL-10 and IL-5 synthesis by P1 came to light (FIG. 10).

FIG. 1: Detection of SEB binding IgE antibodies

Lane 1: molecular weight standard

Lane 2: staining of the SEB molecule with ‘India ink’

Lanes 3-7: detection of SEB-binding IgE antibodies in the serum of patients with atopic eczema

Lanes 8-9: no detection of specific IgE in the serum healthy control persons

Lanes 10-11: negative control with PBS

FIG. 2: Immunoblot with the serum of a healthy control person (NS) and the sera of two patients with atopic eczema (AE) with SEB before (SEB) and after enzymatic digestion with trypsin (SEB+tryp).

MG=molecular weight standard; Ø=control

FIG. 3: Primary amino acid sequence of the SEB molecule. The underlined area corresponds to the first 10 amino acids of the analysed sequence of the 12 kD cleavage product.

FIG. 4: Sequence homology between the IgE binding peptide P1 from SEB and the peptide P2 from the low affine IgE receptor CD23.

FIG. 5: Dot blot with the peptides P1 (SEB peptide) and P2 (CD23 peptide) as well as SEB and purified sera (diluted 1:2) of patients with atopic eczema (trace 2-7). Trace 1 shows the result with an IgG anti-SEB antibody of the sheep. PBS was used as control.

FIG. 6: Proliferation of peripheral blood lymphocytes (n=3) after incubation with P1 and P2 (A) as well as dose-effect curve (10-200 μg peptide) following SEB induced lymphocyte proliferation (B).

FIG. 7: Effects of different concentrations of peptide P1 and P2 following the SEB stimulated intracellular IFNγ (A) and IL-4 production (B). C shows the ratio of IFNγ+Th1 cells to IL-4+Th2 cells.

FIG. 8: Effect of the peptides P1 and P2 following the spontaneous cytokine secretion of blood lymphocytes of healthy candidates (n=3) in vitro. The Th1 cytokines IL-2, IFNγ, TNFα and the Th2 cytokines IL-4, IL-5 and IL-10 were measured.

FIG. 9: Effect of the peptides P1 and P2 following the spontaneous cytokine secretion of blood lymphocytes of patients with atopic eczema (n=3) in vitro. The Th1 cytokines IL-2, IFNγ, TNFα and the Th2 cytokines IL-4, IL-5 and IL-10 were measured.

FIG. 10: Effect of the peptides P1 and P2 following the spontaneous cytokine secretion of peripheral blood lymphocytes of patients with psoriasis vulgaris (n=3) in vitro. The Th1 cytokines IL-2, IFNγ, TNFα and the Th2 cytokines IL-4, IL-5 and IL-10 were measured.

1. Polypeptide with the amino acid sequence indicated in SEQ ID NO:10.

2. Polypeptide characterised in that it exhibits the N-terminal amino acid sequence according to SEQ ID NO:15 and is obtainable by trypsin cleavage of S. aureus enterotoxin B as 12 kD cleavage fragment.

3. Polypeptide characterised in that it is a homologue or derivative of the polypeptide according to claim 1 or 2 which binds to IgE.

4. Polypeptide according to claim 3 characterised in that, in the case of the derivative, one or several L-amino acids are replaced by D-amino acids.

5. Polypeptide according to claims 3 and 4 characterised in that it does not induce the proliferation of T lymphocytes.

6. Polypeptide according to claims 2 to 5 characterised in that it inhibits the synthesis of interferon-gamma stimulated T-lymphocytes in vitro.

7. Polypeptide according to claims 2 to 6 characterised in that it modulates both the stimulated as well as the spontaneous cytokine production of Th1 and Th2 lymphocyte subpopulations in vitro.

8. Polypeptide according to claims 2 to 7 characterised in that it exhibits the amino acid sequence indicated in SEQ ID NO:11.

9. Nucleic acid molecule characterised in that it exhibits a nucleic acid sequence encoding a polypeptide according to claims 1 to 8.

10. Nucleic acid molecule according to claim 9 characterised in that it exhibits the nucleic acid sequence indicated in SEQ ID NO:9.

11. Use of a polypeptide which contains the sequence according to SEQ ID NO:10, a sequence homologous thereto or derived therefrom, the polypeptide binding to IgE, for the production of a pharmaceutical composition for immune modulation.

12. Use of a polypeptide according to claims 1 to 89 for the production of a pharmaceutical composition for immune modulation.

13. Use of a polypeptide which contains the sequence according to SEQ ID NO:10, a sequence homologous thereto or derived therefrom, the polypeptide binding to IgE, for the production of a pharmaceutical composition for inhibiting the cytokine production of lymphocytes.

14. Use of a polypeptide according to claims 1 to 8 for the production of a pharmaceutical composition for inhibiting the cytokine production of lymphocytes.

15. Use according to claims 13 and 14 characterised in that the lymphocytes are T lymphocytes.

16. Use according to claim 15 characterised in that the cytokine is interferon-gamma, interleukin-4 (IL-4), interleukin-5 (IL-5), interleukin-10 (IL-10), interleukin-12 (IL-12) and/or interleukin-13 (IL-13).

17. Use of a polypeptide which contains the sequence according to SEQ ID NO:10, a sequence homologous thereto or derived therefrom, the polypeptide binding to IgE, for the production of a pharmaceutical composition for treating diseases which are associated with an increased production of cytokines and/or an increased level of IgE antibodies.

18. Use of a polypeptide according to claims 1 to 8 for the production of a pharmaceutical composition for treating diseases which are associated with an increased production of cytokines and/or an increased level of IgE antibodies.

19. Use according to claims 17 and 18 characterised in that cytokine is interferon-gamma, interleukin-4 (IL-4), interleukin-5 (IL-5), interleukin-10 (IL-10), interleukin-12 (IL-12) and/or interleukin-13 (IL-13).

20. Use according to claims 17 to 18 characterised in that the disease is atopic eczema, bronchial asthma, allergic rhinoconjunctivitis, psoriasis, rheumatoid arthritis or multiple sclerosis.

21. Use of a polypeptide which contains the sequence according to SEQ ID NO:10, a sequence which is approx. thereto or derived therefrom, the polypeptide binding to IgE, for the production of a pharmaceutical composition for the induction or increasing of a Th1 or Th2 immune response.

22. Use of a polypeptide according to claims 1 to 8 for the production of a pharmaceutical composition for the induction or increasing of a Th1 or Th2 immune response.

23. Use according to claims 21 and 22 for the production of a pharmaceutical composition for the treatment of diseases associated with a Th1 or Th2 immune response.

24. Use according to claims 21 to 23 characterised in that the disease is psoriasis vulgaris, autoimmune uveitis, allergic contact eczema, Behcet's syndrome, diabetes mellitus, Hashimoto's disease, heliobacter pylori infection, Lupus erythematosus, Crohn's disease, multiple sclerosis, organ transplant rejection reaction, psoriasis arthropathica, rheumatoid arthritis and spontaneous, recurrent miscarriages.

25. Use according to claim 24 characterised in that the inflammatory bowel disease is Crohn's disease.

26. Use of a polypeptide which contains the sequence according to SEQ ID NO:10, a sequence homologous thereto or derived therefrom, the polypeptide binding to IgE, for the in vitro inhibition of the interferon-gamma production in human, peripheral T lymphocytes of the blood.

27. Use of a polypeptide according to claims 1 to 8 for the in vitro inhibition of the interferon-gamma production in human, peripheral T lymphocytes of the blood.

28. Use of a polypeptide which contains the sequence according to SEQ ID NO:10, a sequence homologous thereto or derived therefrom, the polypeptide binding to IgE, for the in vitro modulation of the stimulated and the spontaneous cytokine production of Th1 and Th2 lymphocyte subpopulations.

29. Use of a polypeptide according to claims 1 to 8 for the in vitro modulation of the stimulated and the spontaneous cytokine production of Th1 and Th2 lymphocyte subpopulations.

30. Use according to claims 11 to 29 characterised in that the polypeptide is a polypeptide with a sequence derived from SEQ ID NO:10 in which one or several L-amino acids are replaced by D-amino acids.

31. Pharmaceutical composition characterised in that it contains a polypeptide according to claims 1 to 8.

32. Pharmaceutical composition according to claim 31 characterised in that it further contains one or several pharmaceutically compatible auxiliary substances and/or carriers.

33. Process for the production of a polypeptides according to claim 1 characterised in that the nucleic acid sequence shown in SEQ ID NO:9 is introduced into an expression vector, which is introduced into suitable host cells, the host cells are cultivated under conditions suitable for protein expression and the expressed polypeptide which is encoded by the nucleic acid sequence is isolated.

34. Process for the production of a polypeptide according to claim 2 characterised in that enterotoxin B from S. aureus is digested with trypsin and the 12 kD fragment from the reaction batch is subsequently isolated.


SEQUENCE LISTING (15 sequences)

SEQ ID NO: 1 - protein, 18 aa, Artificial Sequence
  synthesized from S. aureus enterotoxin B
HNGNQLDKYRSITVRVFE

SEQ ID NO: 2 - protein, 19 aa, Artificial Sequence
  synthesized from S. aureus enterotoxin B
VRVFEDGKNLLSFDVQTNK

SEQ ID NO: 3 - protein, 19 aa, Artificial Sequence
  synthesized from S. aureus enterotoxin B
VQTNKKKVTAQELDYLTRH

SEQ ID NO: 4 - protein, 19 aa, Artificial Sequence
  synthesized from S. aureus enterotoxin B
YLTRHYLVKNKKLYEFNNS

SEQ ID NO: 5 - protein, 19 aa, Artificial Sequence
  synthesized from S. aureus enterotoxin B
EFNNSPYETGYIKFIENEN

SEQ ID NO: 6 - protein, 20 aa, Artificial Sequence
  synthesized from S. aureus enterotoxin B
IENENSFWYDMMPAPGDKFD

SEQ ID NO: 7 - protein, 20 aa, Artificial Sequence
  synthesized from S. aureus enterotoxin B
GDKFDQSKYLMMYNDNKMVD

SEQ ID NO: 8 - protein, 20 aa, Artificial Sequence
  synthesized from S. aureus enterotoxin B
NKMVDSKDVKIEVYLTTKKK

SEQ ID NO: 9 - DNA, 27 bp, Artificial Sequence
  synthesized from S. aureus enterotoxin B
caagaattagattacctaactcgtcac

SEQ ID NO: 10 - protein, 9 aa, Artificial Sequence
  synthesized from S. aureus enterotoxin B
QELDYLTRH

SEQ ID NO: 11 - protein, 9 aa, Artificial Sequence
  synthesized from S. aureus enterotoxin B
QEEDFLTLH

SEQ ID NO: 12 - DNA, 1712 bp, S. aureus
  sig_peptide (244)...(324)
  mat_peptide (325)...(1041): Enterotoxin B
gaactaggtagaaaaataattatgagaaaacactatgttgttaaagatgttttcgtatataagtttaggtgatgtatagttacttaattttaaaagcataacttaattaatataaataacatgagattattaaatataattaagtttcttttaatgtttttttaattgaatatttaagattataacatatatttaaagtgtatctagatactttttgggaatgttggataaaggagataaaaaatgtataagagattatttatttcacatgtaattttgatattcgcactgatattagttatttctacacccaacgttttagcagagagtcaaccagatcctaaaccagatgagttgcacaaatcgagtaaattcactggtttgatggaaaatatgaaagttttgtatgatgataatcatgtatcagcaataaacgttaaatctatagatcaatttctatactttgacttaatatattctattaaggacactaagttagggaattatgataatgttcgagtcgaatttaaaaacaaagatttagctgataaatacaaagataaatacgtagatgtgtttggagctaattattattatcaatgttatttttctaaaaaaacgaatgatattaattcgcatcaaactgacaaacgaaaaacttgtatgtatggtggtgtaactgagcataatggaaaccaattagataaatatagaagtattactgttcgggtatttgaagatggtaaaaatttattatcttttgacgtacaaactaataagaaaaaggtgactgctcaagaattagattacctaactcgtcactatttggtgaaaaataaaaaactctatgaatttaacaactcgccttatgaaacgggatatattaaatttatagaaaatgagaatagcttttggtatgacatgatgcctgcaccaggagataaatttgaccaatctaaatatttaatgatgtacaatgacaataaaatggttgattctaaagatgtgaagattgaagtttatcttacgacaaagaaaaagtgaaattatattttagaaaagtaaatatgaagagttagtaattaaggcaggcacttatagagtacctgccttttctaatattatttagttatagttatttttgttatatctctctgatttagcattaaccccttgttgccattatagttttcaccaactttagctgaaattgggggatcatttttatctttactatggatagttactgtgtcgccgtttttaacgatttgtttctcttttaatttgtcagttaattttttccatgcatcatttgcgtcaaacctatttccatttggatttattcttgacaaatcaattcttttaacactatcggtattaatcggcttgttattaaaattactaagttcatctaaatcagctgtacccgtaatactactttcgccaccattatttaaattgtacgtaacaccaactgtctcatttgctgttttatcgataatatttgcttctttcaaagcatctcttacatttttccataagtctctatctgttatttcagaagcctttgcaacgttattaataccattataatttgaagaagaatgaaaacctgaacctactgttgttaaaactaaagcacttgctatcaatgttcttgttaatagttttttattcattttattttctcctataacttatttgcaatcgat

SEQ ID NO: 13 - protein, 266 aa, S. aureus
MYKRLFISHVILIFALILVISTPNVLAESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 14 - protein, 239 aa, S. aureus
ESQPDPKPDELHKSSKFTGLMENMKVLYDDNHVSAINVKSIDQFLYFDLIYSIKDTKLGNYDNVRVEFKNKDLADKYKDKYVDVFGANYYYQCYFSKKTNDINSHQTDKRKTCMYGGVTEHNGNQLDKYRSITVRVFEDGKNLLSFDVQTNKKKVTAQELDYLTRHYLVKNKKLYEFNNSPYETGYIKFIENENSFWYDMMPAPGDKFDQSKYLMMYNDNKMVDSKDVKIEVYLTTKKK

SEQ ID NO: 15 - protein, 10 aa, Artificial Sequence
  N-terminal sequence of the 12kD trypsin cleavage fragment from S. aureus enterotoxin B
KVTAQELDYL